(19) United States
(12) Patent Application Publication
(10) Pub. No.: US 2024/0359026 A1
(43) Pub. Date: Oct. 31, 2024

(54) Biometric Electromagnetic Scan Skin Bodysuit

(71) Applicant: James Edward Davis, JR., Boca Raton, FL (US)

(72) Inventor: James Edward Davis, JR., Boca Raton, FL (US)

(21) Appl. No.: 18/322,164

(22) Filed: May 23, 2023

Related U.S. Application Data
(63) Continuation-in-part of application No. 29/875,005, filed on Apr. 27, 2023.

(51) Int. Cl. A61N 2/00; A61B 5/00; A61H 9/00

(52) U.S. Cl. CPC A61N 2/002; A61B 5/6804; A61H 9/0092; A61H 2201/0207; A61H 2201/0214; A61H 2201/10; A61H 2201/105; A61H 2201/1604; A61H 2201/1619; A61H 2201/1635; A61H 2201/164; A61H 2201/165; A61H 2230/201; A61H 2230/305

(57) ABSTRACT

A wearable technology, referred to herein as biometric bodysuit, which integrates biometric sensors, electromagnetic stimulation, and wireless control modules having the capacity to communicate with an interactive user interface. The body is comprised of at least one segment covering at least a portion of a body of a wearer. The segments include an arm covering, a leg covering, a torso covering, footwear, handwear, headwear, and any combination thereof. The segments include a series of control modules that relay real-time biometric data to a main control module, wherein the data is then relayed to a server and a user interface. The user interface permits wearers of the bodysuit and healthcare professionals to adjust the functionality of the bodysuit in accordance to what is the necessary therapy required by the wearer.

### FIELD OF THE INVENTION

The present invention relates generally to a wearable device for measuring, detecting, and assisting bioelectric signals in the body. More specifically, the present invention is a bodysuit covering at least a portion of the body of a wearer, providing biometric data collection, measurement, analysis, and diagnostics of the wearer.

### BACKGROUND OF THE INVENTION

Advancements in wearable technology have led to significant improvements in monitoring and enhancing human performance. Biometric sensors embedded in wearable devices have enabled real-time tracking of vital signs, body movements, and physical activities. Similarly, muscle and nerve stimulation techniques have been employed to aid in muscle recovery, rehabilitation, and the overall enhancement of human performance as it pertains to physiological processes. However, it has been found in the prior art that the integration of these technologies into a single comprehensive solution remains absent. The biometric electromagnetic bodysuit described herein seeks to combine biometric sensing and diagnostics capabilities with muscle enhancement, nerve stimulation, and pneumatic functionalities within a modular garment, capable of providing real-time information pertaining to a wearer's biometrics via an interactive user interface that controls the multiple functionalities of the bodysuit.

Biometric sensors play a crucial role in capturing physiological and biomechanical data from the human body. Existing wearable devices incorporate sensors such as heart rate monitors, accelerometers, gyroscopes, and temperature sensors to provide insights into the wearer's health and physical activity. These sensors can monitor heart rate, respiratory rate, body temperature, and motion patterns, allowing for the analysis of fitness levels, stress levels, and sleep quality. The placement of these sensors on traditional wearable devices does not produce an encompassing reading of a wearer's biometrics, nor do these current devices provide a means to address the problem using the same device. The electromagnetic bodysuit provides a solution to this problem by integrating biometric sensors directly into a multi-layered material capable of transmitting electrical signals throughout the suit, relaying data in real time to a control unit, ensuring accurate real-time data collection and the ability to address biometric and physiological issues as they arise.

Electromagnetic muscle and nerve stimulation techniques have been widely used in various fields, including physical therapy, sports training, and pain management. Electromagnetic stimulation (EMS) of muscles can help strengthen weakened muscles, enhance blood circulation, and promote muscle recovery. Similarly, nerve stimulation can alleviate pain, improve muscle control, and facilitate rehabilitation after injuries. Existing EMS devices often require cumbersome setups with electrodes attached to the skin at specific locations, limiting the application and effectiveness of the therapy, while additionally restricting the mobility of the wearer, as it is now made difficult to move normally without the risk of an electrode coming detached. The electromagnetic bodysuit overcomes this limitation by integrating electromagnetic stimulation components directly into the conductive material which are controlled by a series of wirelessly communicative control modules, providing targeted stimulation and physiological process enhancement to specific muscle groups and nerves via a centralized unit.

### SUMMARY OF THE INVENTION

The electromagnetic bodysuit described in this patent is a next-generation wearable garment that combines biometric sensing capabilities and EMS technology. The suit is composed of a multi-layered conductive Kevlar and graphene material with carbon nanotubes, a control module, biometric sensors, and electromagnetic stimulation components. The biometric sensors accurately monitor vital signs, body movements, and physical activities in real-time, providing valuable data for health tracking, analysis, and diagnostics. The electromagnetic stimulation components emit controlled electrical impulses to target specific muscle groups and nerves, facilitating muscle recovery, repair, and rehabilitation. The biometric sensors integrated into the electromagnetic bodysuit are strategically positioned to ensure accurate and consistent data collection from key areas of the body of the wearer. The sensors continuously monitor vital signs, including heart rate, respiratory rate, body temperature, and motion patterns. This data is processed and analyzed to provide real-time feedback on the wearer's health status and performance levels. The electromagnetic stimulation components embedded within the material of the bodysuit enable targeted muscle and nerve stimulation. These components generate controlled electrical impulses that stimulate specific muscle groups and nerves, promoting muscle activation, improving muscle coordination, and facilitating rehabilitation processes. By combining real-time monitoring of vital signs and physical activity with targeted stimulation, the electromagnetic bodysuit provides a holistic approach to optimize human performance and rehabilitation. Athletes can leverage the bodysuit's capabilities for sports training, injury prevention, and recovery, while individuals undergoing physical therapy can benefit from personalized stimulation protocols tailored to their specific needs. Moreover, the adaptability, modularity, and flexibility of the electromagnetic bodysuit allow for seamless integration into various daily activities, making it a versatile and wearer-friendly solution.

In addition to its biometric sensing and muscle/nerve stimulation capabilities, the electromagnetic bodysuit described in this patent incorporates an interactive user interface that allows wearers to control and customize the suit's functions. The user interface provides a seamless and intuitive way to interact with the suit, empowering individuals to optimize their training, rehabilitation, and experience using the suit. The interactive user interface can be accessed through devices such as smartphones and dedicated wearable devices. By connecting to the suit via wireless communication, healthcare professionals and wearers gain access to the biometric data and control over the suit's functions, thus enhancing its usability and adaptability.

The present invention incorporates a modular design that enables the seamless integration of at least one segment, including arm sleeves, a torso vest, leg sleeves, a helmet, gloves, and boots. Each modular section is designed to cater to specific body parts and functionalities, providing targeted muscle and nerve stimulation, as well as biometric sensing. These modular sections are interconnected wirelessly via a series of control modules, forming a cohesive system that communicates data and diagnostic processes throughout the segments.

The arm sleeves of the electromagnetic bodysuit are designed to wrap around a portion of an arm of the wearer, from the shoulders to the wrists. These sleeves contain a control module and biometric sensors that monitor vital signs and capture movement patterns of the arm extremity of the wearer. They also incorporate electromagnetic stimulation components that deliver targeted electrical impulses to the arm muscles via the conductive material, aiding in muscle activation, coordination, and rehabilitation. Similarly, the vest module encompasses the torso and contains biometric sensors strategically placed to monitor heart rate, respiratory rate, and body temperature. It also integrates stimulation components to stimulate muscles in the core, chest, and back of the wearers, assisting in facilitating biometric and physiological functions of the wearer. The leg sleeves provide stimulation and monitoring capabilities for the lower body. Biometric sensors embedded in the leg sleeves capture data related to movement patterns, muscle activation and integrity, and nerve imbalances. The stimulation components target specific leg muscles, facilitating muscle rehabilitation, enhancing performance, and aiding in correction. The helmet module integrates biometric sensors to monitor and provide therapy to the head of the wearer, including psychological therapy, and potentially treating eye diseases. Additionally, the helmet module incorporates stimulation components that provide targeted stimulation to specific nerves or muscles in the head and neck region. The gloves and boots modules of the electromagnetic bodysuit are designed to enhance hand and foot functionalities, respectively. Biometric sensors integrated into the gloves capture data related to hand movements, grip strength, and dexterity. The gloves also incorporate stimulation components that can aid in hand muscle and nerve rehabilitation. The boots, on the other hand, contain sensors that monitor biometric data within the foot of a wearer.

The wireless communication enables seamless synchronization between the segments of the suit and the interactive user interface, ensuring coordinated stimulation and data sharing. The wireless communication between the modular sections of the electromagnetic bodysuit enables seamless synchronization, data sharing, and coordination. It ensures that the various components work together as a unified system, providing targeted stimulation and collecting comprehensive biometric data. The wireless connectivity allows for real-time adjustments, personalized control, and adaptive stimulation patterns based on the wearer's needs and goals.

### BRIEF DESCRIPTION OF THE DRAWINGS

FIG. 1 is a front view of the present invention.

FIG. 2 is a rear view of the present invention.

FIG. 3 is a view of the arm sleeve of the present invention.

FIG. 4 is a front view of one embodiment of the leg sleeve of the present invention.

FIG. 5 is a perspective view of one embodiment of the leg sleeve of the present invention.

FIG. 6 is a perspective view of the torso cover of the present invention.

FIG. 7 is a perspective view of the helmet of the present invention wherein the helmet is being worn by the wearer.

FIG. 8 is a perspective view of the footwear of the present invention.

FIG. 9 is a front view of the glove of the present invention.

FIG. 10 is a diagram showing the hardware that comprises the main central control modules of the present invention.

FIG. 11 is a diagram showing the hardware that comprises the primary central control modules of the present invention.

FIG. 12 is a pictorial representation of the communication between the bodysuit, the network, and a wearable device of the present invention.

FIG. 13 is a pictorial representation of the communication between the bodysuit, the network, and a mobile application of the present invention.

FIG. 14 is a dissected view of the multilayered material of the present invention.

FIG. 15 is a flow diagram of the communication path between the bodysuit, the network, and the user interface of one embodiment of the present invention comprising a mobile application.

FIG. 16 is a flow diagram of the communication path between the bodysuit, the network, and the user interface of an alternate embodiment of the present invention comprising a wearable device.

FIG. 17 is a diagram displaying the plurality of functions of the present invention.

FIG. 18 is a diagram displaying the first function of the present invention.

FIG. 19 is a diagram displaying the second function of the present invention.

FIG. 20 is a diagram displaying the eighth function of the present invention.

### DETAIL DESCRIPTIONS OF THE INVENTION

All illustrations of the drawings are for the purpose of describing selected versions of the present invention and are not intended to limit the scope of the present invention.

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

Other technical advantages may become readily apparent to one of ordinary skill in the art after review of the following figures and description. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

Unless otherwise indicated, the drawings are intended to be read together with the specification, and are to be considered a portion of the entire written description of this invention. As used in the following description, the terms “horizontal”, “vertical”, “left”, “right”, “up”, “down” and the like, as well as adjectival and adverbial derivatives thereof (e.g., “horizontally”, “rightwardly”, “upwardly”, “radially”, etc.), simply refer to the orientation of the illustrated structure as the particular drawing figure faces the reader. Similarly, the terms “inwardly,” “outwardly” and “radially” generally refer to the orientation of a surface relative to its axis of elongation, or axis of rotation, as appropriate.

The present disclosure includes many aspects and features. Moreover, while many aspects and features relate to, and are described in the context of an electromagnetic biometric bodysuit 1, embodiments of the present disclosure are not limited to use only in this context.

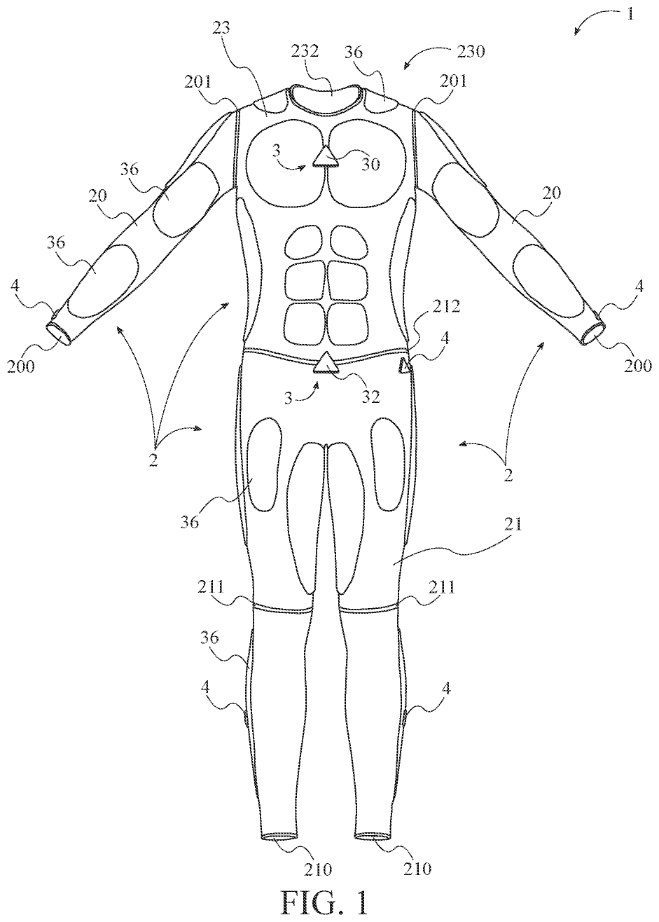

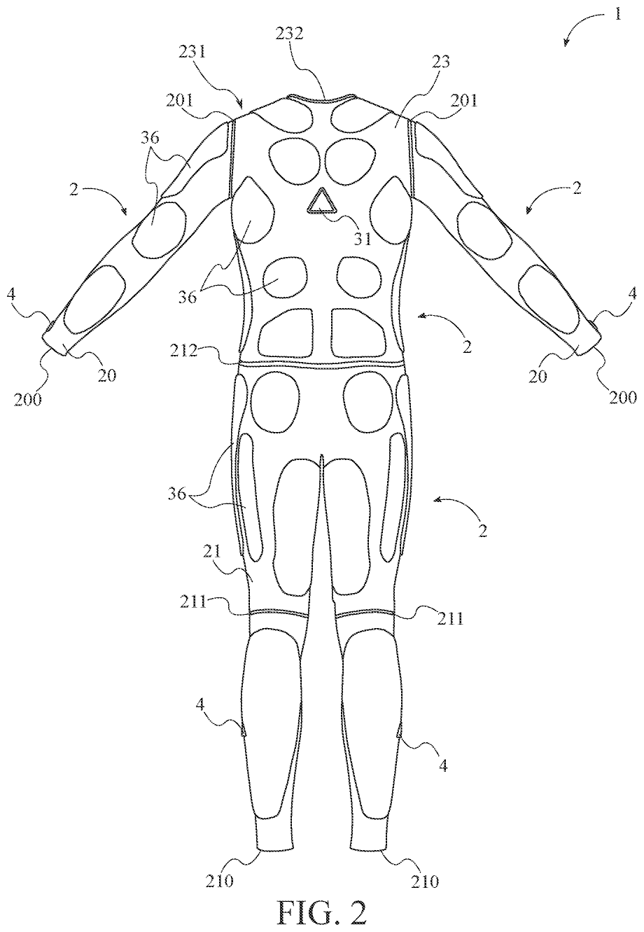

As shown in FIG. 1 and FIG. 2, the present invention is an electromagnetic bodysuit 1, referred to herein as a bodysuit 1, comprising an at least one segment 2, wherein said segment 2 covers at least a portion of a body of a wearer. In the preferred embodiments of the present invention, the segment 2 may include an at least one arm sleeve 20, an at least one leg sleeve 21, a torso cover 23, a helmet 24, an at least one footwear 25, an at least one glove 26, and a combination thereof, as shown in FIGS. 1-9. In the preferred embodiment of the present invention, the segments 2 of the bodysuit 1 are composed of a multilayered material 6. In an embodiment of the present invention wherein a plurality of segments 2 are worn by the wearer, the segments 2 are modular and may comprise a shared connection point, also referred herein as a seam.

In the preferred embodiment of the present invention, the bodysuit 1 as shown in FIG. 1 and FIG. 2, comprise the plurality of segments 2, whereby two arm sleeves 20 are coupled adjacent to the torso cover 23 at a shared seam proximate a shoulder 201 of a wearer. Furthermore in the aforementioned embodiment, the torso cover 23, is coupled adjacent to the leg sleeves 21, whereby the leg sleeves 21 cover both legs of the wearer.

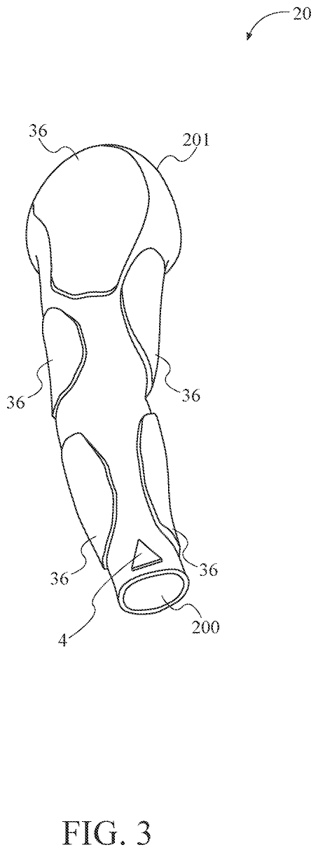

As shown in FIG. 3, the arm sleeve 20 comprises a wrist opening 200, a shoulder seam 201, a primary central control module 4, and a plurality of input/output devices 36 (I/O devices). In the preferred embodiment of the present invention, the arm sleeve 20 is attached to the torso cover 23 at the shoulder seam 201.

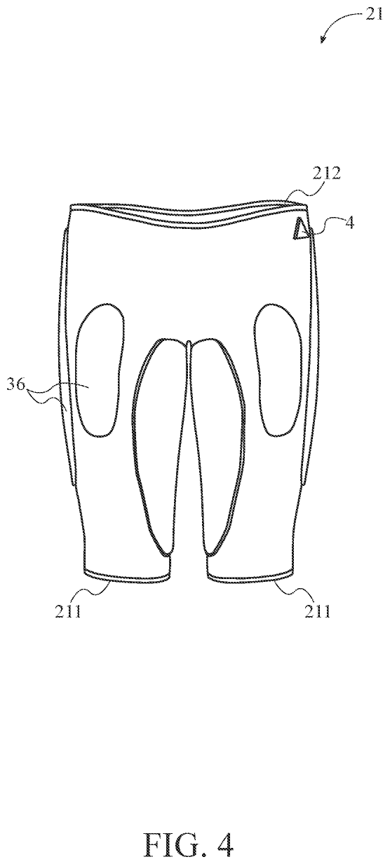

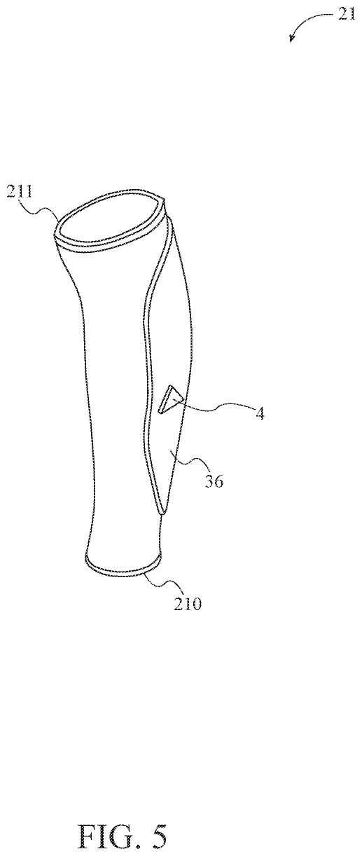

As shown in FIG. 4, in the preferred embodiment of the present invention, the leg sleeve 21 comprises a plurality of I/O devices 36, and a primary central control module 4. In some embodiments, wherein the leg sleeve 21 further comprises a waist seam, the waist seem is proximate a waist of the wearer. In some embodiments of the present invention, the leg sleeve 21 comprises an plurality of openings 211 at a location below the waist 212 when in the worn configuration. In some embodiments of the present invention, the plurality of openings is proximate a knee 211 of the wearer. In an alternative embodiment of the present invention, the leg sleeve 21 comprises an opening proximate the knee 211 and an opening proximate an ankle 210 of the wearer, as shown in FIG. 5.

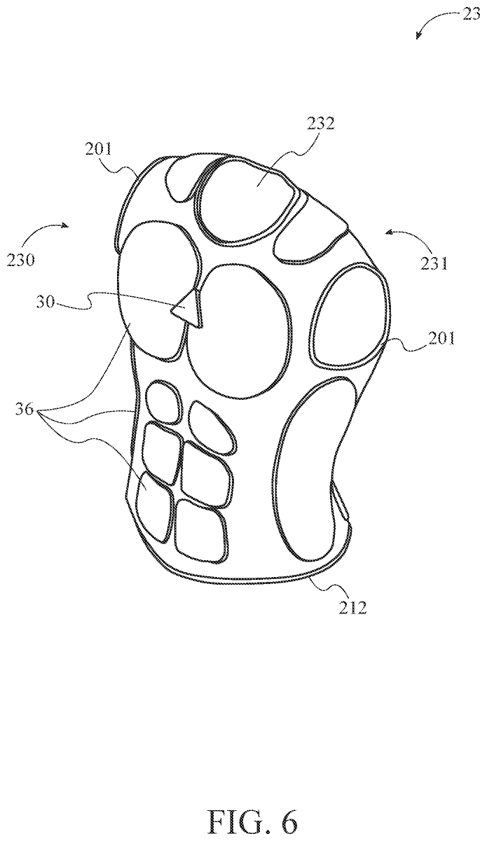

In some embodiments of the present invention, as shown in FIG. 6, the torso cover 23 comprises a plurality of I/O devices 36, an at least one main central control module 3, and a neck opening 232. In the preferred embodiment of the present invention, the torso cover 23 comprises a front facing side 230 and a rear facing side 231 wherein a main central control module 3 is coupled to the front facing side 230 proximate a chest 30 of the wearer, in addition to a main central control module 3 coupled to the rear facing side 231 of the torso cover 23 proximate the center of a back 31 of the wearer. In some embodiments of the present invention, the torso cover 23 comprises an opening proximate the shoulder 201 of the wearer, whereby the arm sleeve 20 is coupled.

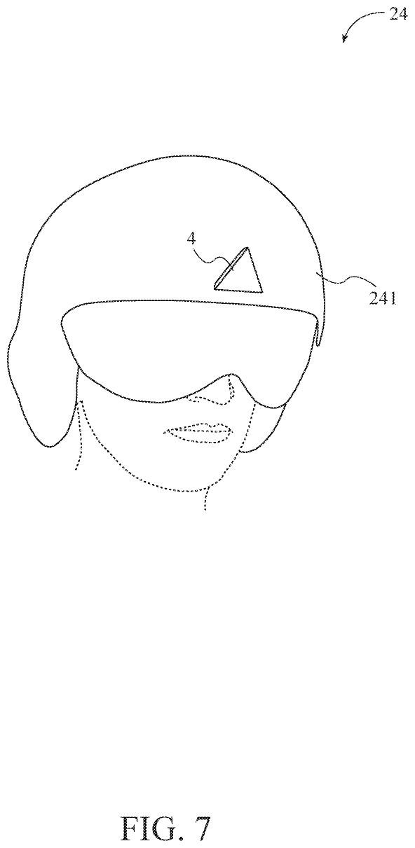

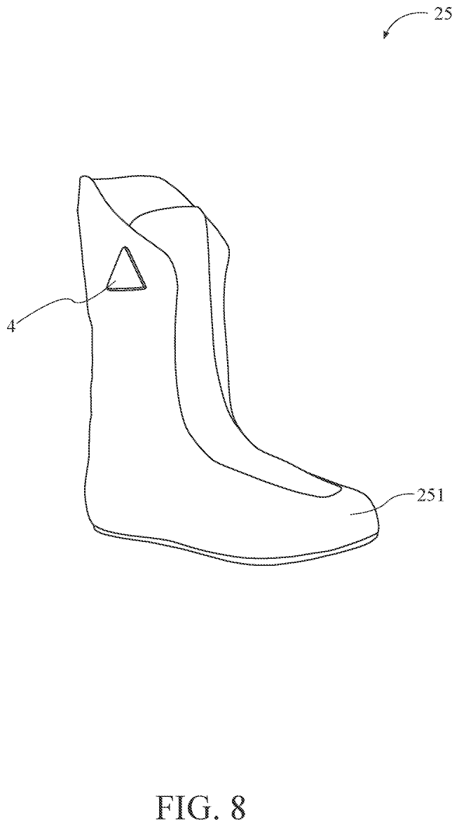

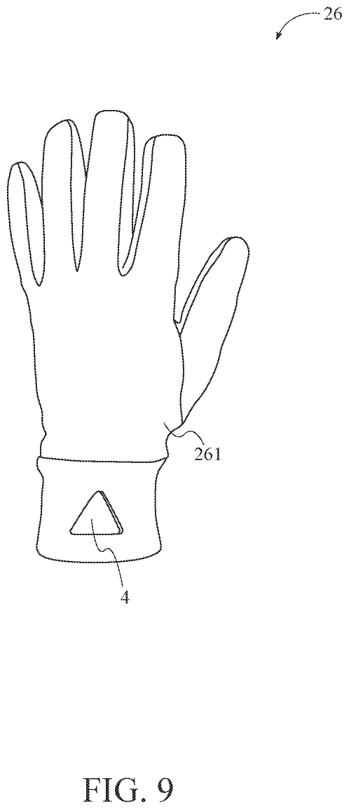

In some embodiments of the present invention, the bodysuit 1 further comprises a helmet 24 wherein said helmet 24 comprises a primary central control module 4, as shown in FIG. 7. In some embodiments of the present invention, the bodysuit 1 further comprises a footwear 25, wherein said footwear 25 comprises a primary central control module 4, as shown in FIG. 8. Additionally, in some embodiments of the present invention, the bodysuit 1 comprises a glove 26, as shown in FIG. 9, comprising a primary central control module 4.

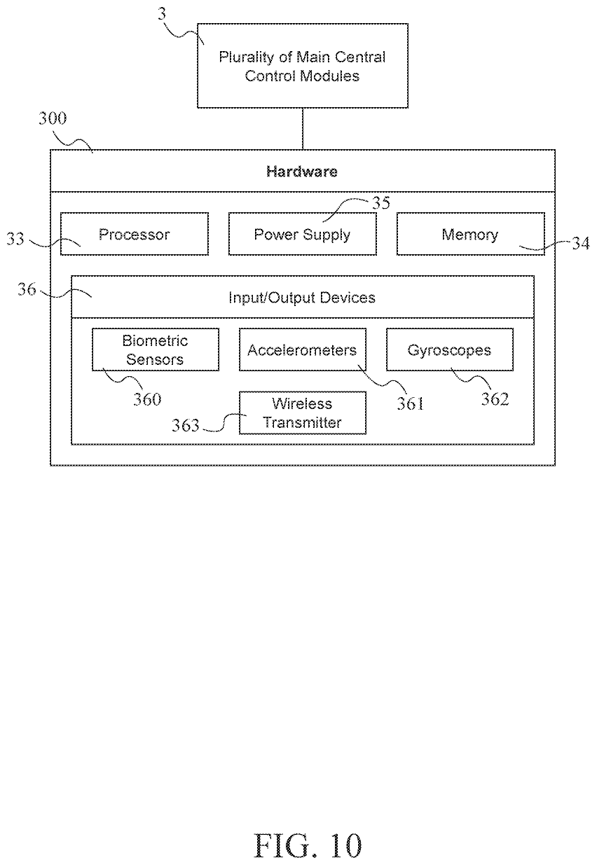

As shown in FIG. 10, in the preferred embodiment of the present invention, the bodysuit 1 further comprises an at least one main central control module 3. In the preferred embodiment of the present invention, the bodysuit 1 comprises three main central control modules 3 located proximate a chest 30 of a wearer, proximate a waist 32 of a wearer, and on a back 31 of a wearer. The main central control modules 3 are coupled to the segments 2 of the bodysuit 1. The main central control module 3 is a computer device comprising a processor 33 and a power device 35. The power device 35 may include a device including a battery, a supercapacitor, and devices known to one in the art capable of storing and releasing an electronic charge. In some embodiments of the present invention, the main central control module 3 further comprises a memory storage device 34.

Furthermore, in the preferred embodiment of the present invention the bodysuit 1 comprises at least one electronic hardware device wherein said hardware is connected, either through a wireless communication or via a wired connection, to the main central control module 3. In the aforementioned embodiment, the hardware referred to therein is an input/output device 36 (I/O device) comprising at least one of the following including a biometric sensor 360, an accelerometer 361, a gyroscope 362, a wireless transmitter 363, and a combination thereof. The biometric sensor 360 is a device that has the capability to perform measurements on physiological and biological functions or a wearer including but not limited to a body temperature, a heart rate, an electrical signal, a respiration rate, a blood pressure, an oxygen saturation, and a combination thereof. The wireless transmitter 363, in the case of the present invention, is intended to include a wireless transmitter and a wireless receiver.

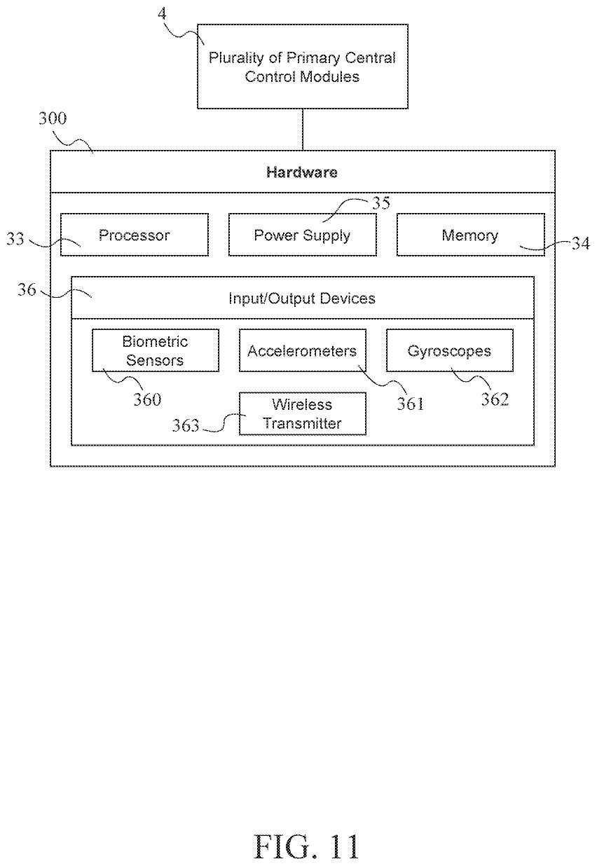

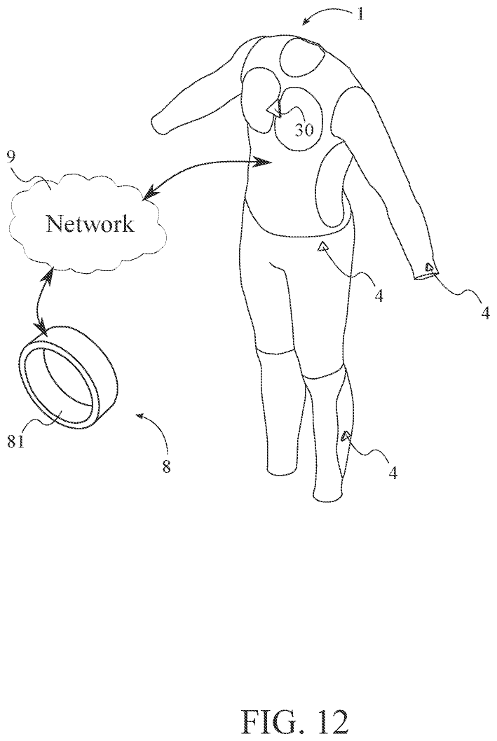

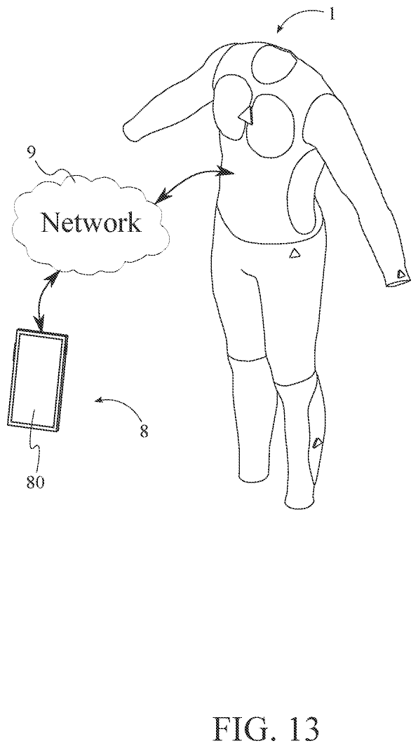

In the preferred embodiment of the present invention, the segments 2 of the bodysuit 1 further comprise an at least one primary central control module 4, as shown in FIG. 11. The primary central control module 4 comprises the same components as the main central control module 3. In the preferred embodiment of the present invention, the I/O devices 36 receive information from the body of the wearer, and transmit said information to the main central control module 3. The primary central control modules 4 are coupled to the segments 2 of the bodysuit 1. Additionally, in the preferred embodiment of the present invention, as shown in FIG. 12 and FIG. 13, the bodysuit 1 further comprises a user interface 8 that communicates with a server 90. In the aforementioned embodiment of the present invention, the bodysuit 1, more specifically the main central control module 3 and the user interface 8 communicate with a server 90 which comprises an external network 9.

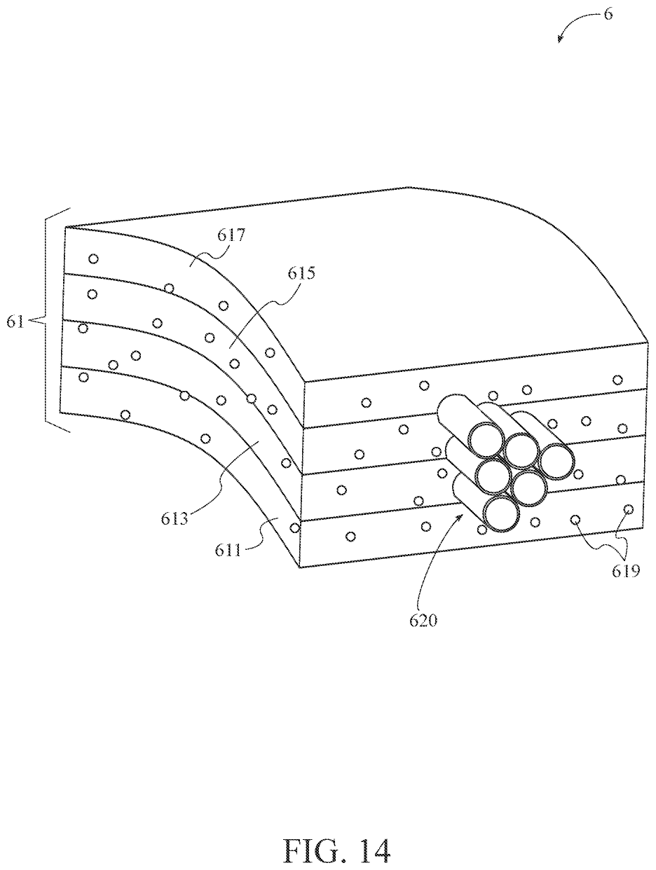

The segments 2 of the bodysuit 1 are composed of the multilayered material 6 wherein the multilayered material 6 is composed of a plurality of layers 61 comprising a first layer 611, a second layer 613, a third layer 615, and a fourth layer 617, as shown in FIG. 14. The first layer 611 is the innermost layer, wherein said layer is proximate the body of the wearer. In the preferred embodiment of the present invention, the first layer 611 of the multilayered material 6 is composed of a nylon tricot material. The second layer 613, interposed between the first layer 611 and the third layer 615 of the multilayered material 6 is composed of a spandex material. The third layer 615 of the multilayered material 6, wherein said layer is interposed between the second layer 613 and the fourth layer 617, is composed of a urethane coated nylon. In the preferred embodiment of the present invention, the third layer 615 is interposed between the second layer 613 and the fourth layer 617, however the third layer 615 has the ability to expand and contract, decreasing and increasing the space, and thus the pressure applied to, the adjacent layers. The fourth layer 617, wherein said layer is the outermost layer and adjacent to the third layer 615 of the multilayered material 6, is composed of a Kevlar and graphene material. In alternative embodiments of the present invention, the fourth layer 617 further comprises polyethylene terephthalate. The fourth layer 617 is relatively non-flexible, providing a barrier, wherein said barrier inhibits the third layer 615 from exerting an outward force, and rather applying an inward force towards the body of the wearer; this process being referred to herein as pressurization. Additionally, the fourth layer 617, comprising graphene, is an electrically conductive layer permitting electrical signals to travel throughout the suit. Furthermore, the multilayered material 6 comprises a plurality of carbon nanotubes 620 suspended in polyethylene glycol 619. In the preferred embodiment of the present invention, the multilayered material 6 comprises six carbon nanotubes 620. In alternative embodiments the multilayered material 6 comprises seven carbon nanotubes 620.

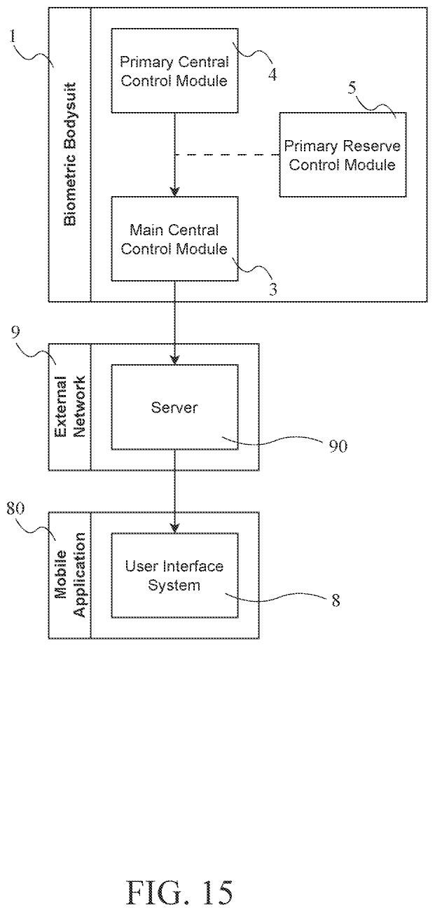

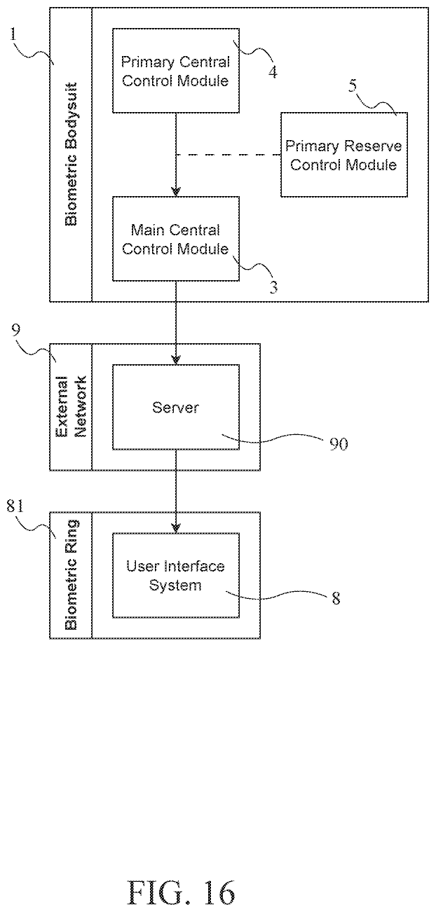

In the preferred embodiment of the present invention, the bodysuit 1 further comprises an at least one primary reserve control module 5. The primary reserve control module 5 is a secondary control module that acts as a backup to the primary central control module 4. As shown in FIG. 15 and FIG. 16, the bodysuit 1, after receiving data from the I/O devices 36, communicates data from the primary central control module 4 to the main central control module 3, to the server 90, and finally to the user interface 8, wherein said interface is a mobile application 80 or a biometric ring 81. The user interface 8 allows a wearer and a health care professional to access data collected and communicated by the bodysuit 1.

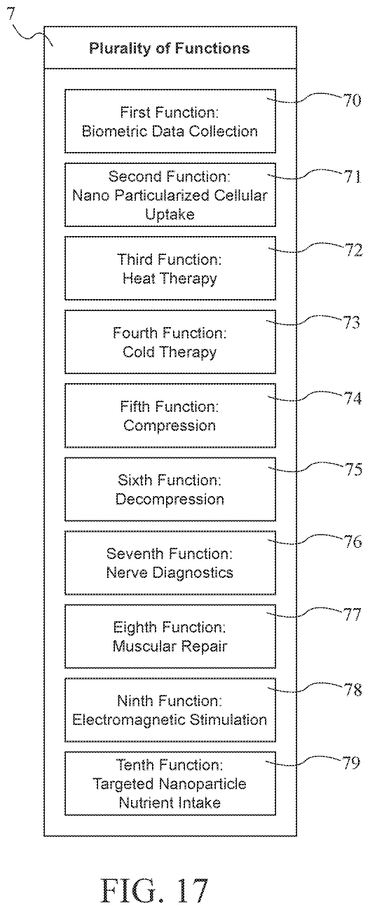

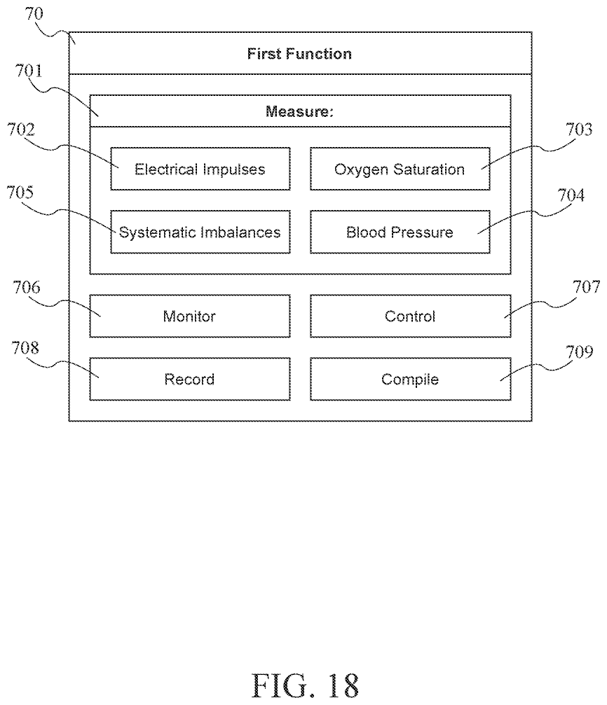

Using the user interface 8, the wearer may then enable a plurality of functions 7, as shown in FIG. 17. In the preferred embodiment of the present invention, the bodysuit 1 comprises a first function 70 wherein said first function 70 is a biometric data collection process. In the biometric data collection process, as shown in FIG. 18, the biometric sensors 360 measure biometric information of the wearer, including but not limited to electrical impulses 702, oxygen saturation 703, systematic imbalances 705 within the muscle and nervous system, and blood pressure 704. Further, in the first function 70, biometric information is monitored 706 over a period of time, controlled 707, recorded 708, and compiled 709. When monitoring the biometric information, the I/O devices 36 collect at least two data points over the span of an interval and compare a first biometric data point with a second biometric data point. Comparing the data points, the bodysuit 1 communicates the data to the wearer via the user interface 8 wherein the wearer may make adjustments to the bodysuit 1, thus allowing the wearer to control 707 the biometrics of the body of the wearer. Additionally, the data collected by the bodysuit 1 is then communicated to a server 90 wherein the server 90 records and stores 708 and compiles 709 the data.

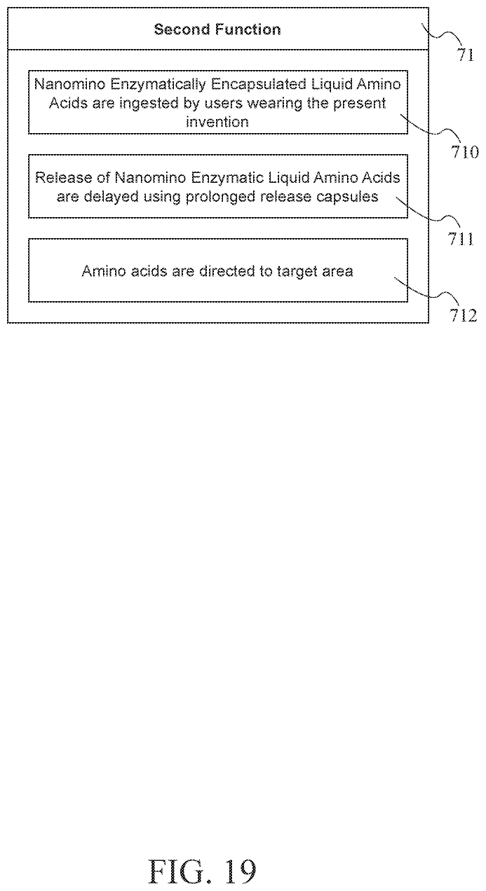

In one embodiment of the present invention, the bodysuit 1 further comprises a second function 71. The second function 71 is a process wherein the biometric sensors 360 of the bodysuit 1 facilitate the uptake of nanoparticularized amino acids, referred herein as cellular uptake of enzymatically encapsulated liquid amino acids, as shown in FIG. 19. During the process of cellular uptake of enzymatically encapsulated liquid amino acids, the wearer ingests encapsulated amino acids, also referred to as nanominos, while wearing the present invention. Upon the release of the amino acids, the biometric sensors 360 of the bodysuit 1 determine areas of the body that are in need of amino acids, such as an area of the body wherein the wearer of the bodysuit 1 has a muscle in need of repair. Upon determining the area of the body of the wearer in need of repair, an electrical signal is sent to the I/O device 36 in a proximate area and emits an electromagnetic signal, simulating a natural process of a human body, thus directing amino acids directly to said area of the body.

In an additional embodiment of the present invention, the present invention further comprises a third function 72, wherein said function is a process referred to as heat therapy. In the heat therapy process, the wearer increases a temperature of the bodysuit 1. Furthermore, in an additional embodiment of the present invention, the bodysuit 1 further comprises a fourth function 73, wherein said process is a process referred herein as cold therapy. In the cold therapy process, the bodysuit 1 restricts circulation to an area of the body of the wearer to simulate the sensation of a temperature decrease within the bodysuit 1. In some embodiments of the present invention, the bodysuit 1 further comprises a fifth function 74 wherein said fifth function 74 is a compression process. The compression process is a process wherein via an actuator, the third layer 615 of the multilayered material 6 expands, similarly to an sphygmomanometer, applying an inward force to the bodysuit 1 and thus compressing a portion of the body of a wearer. In some embodiments of the present invention, the bodysuit 1 further comprises a sixth function 75 wherein said function 75 is a decompression process. In said decompression process, the third layer 615 of the multilayered material 6 contracts via a release of pressure, reducing the applied inward pressure to the portion of the body of the wearer. In some embodiments of the present invention, the bodysuit 1 further comprises a seventh function 76. The seventh function 76 of the present invention is a nerve diagnostics function wherein the biometric sensors 360 detect, measure, and address electrical impulses from nerves within the body of the wearer. The biometric sensors 360 within the segments 2 of the bodysuit 1 collect electrical signals from various areas of the body of the wearer to determine imbalances within nerve diagnostics. By measuring the resistance of firing nerves, the bodysuit 1 compares the differences, compiles the data, and presents the findings in a user readable format via the user interface 8.

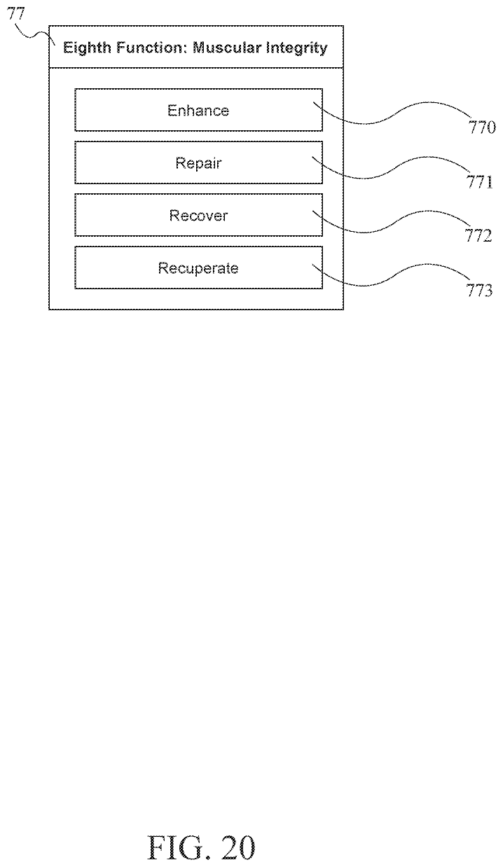

In some embodiments of the present invention, the bodysuit 1 comprises an eighth function 77. In the preferred embodiment of the present invention, the eighth function 77 is a process whereby the bodysuit 1 facilitates the repair of muscular integrity, as shown in FIG. 20. In the aforementioned muscular repair process, the bodysuit 1 via the I/O devices 36, the user interface 8, and the processor 33, assists the wearer, by providing feedback regarding muscular integrity whereby the suit facilitates the enhancement 770, the repair 771, the recovery 772, and the recuperation 773 of muscular integrity. Additionally, in some embodiments of the present invention, the bodysuit 1 further comprises a ninth function 78. In some embodiments of the present invention the ninth function 78 of the present invention is a process wherein the suit emits an electromagnetic pulse to the body of the wearer, stimulating the body in predetermined areas of the body. The aforementioned process whereby electromagnetic pulses are emitted to the body of the wearer, referred herein as the process of electromagnetic stimulation, uses the conductivity of the multilayered material 6, the I/O devices 36, the processor 33, the main central control module 3, and the user interface 8 to send signals and collect feedback from the wearer. The electromagnetic signals stimulate the body whereby said stimulation facilitates improved circulation, mobility, healing, and recovery. In the process of muscular repair, the electromagnetic stimulation sends pulses to a targeted area to stimulate the nerves and muscles in a manner similar to that in nature, whereby said stimulation facilitates muscle enhancement 770, repair 771, recovery 772, and recuperation 773. In further embodiments of the present invention, the bodysuit 1 comprises a tenth function 79. The tenth function 79 is a process whereby the bodysuit 1 assists in the uptake of targeted nanoparticle nutrients. In the uptake of nanoparticle nutrients, the bodysuit 1 may track and direct medical products including medicinal supplements.

Although the plurality of functions 7 have been assigned numbers, the specification is not to limit the processes to the respective number. The functions 7 have been numbered as a means of concisely detailing each function.

Although the invention has been explained in relation to its preferred embodiment, it is to be understood that many other possible modifications and variations can be made without departing from the spirit and scope of the invention.

What is claimed is:

1. A bodysuit comprising:
at least one segment wherein said segment covers at least a portion of a body of a wearer;
the segment further comprising at least one main central control module wherein said main central control module is coupled to the segment of the bodysuit;
the segment further comprising at least one hardware device wherein said hardware device is an input/output device such as a biometric sensor, an accelerometer, a gyroscope, and a wireless transmitter;
the hardware device coupled to the segment of the bodysuit, wherein said hardware device has the capability to wirelessly transmit data between said hardware device and the control module.

2. The bodysuit as claimed in claim 1 wherein the at least one segment comprises at least one of the following including:
an arm sleeve comprising a first opening proximate a forearm of a wearer and a second opening proximate a shoulder of the wearer;
a leg sleeve comprising a first opening proximate a lower portion of a leg of the wearer and a second opening proximate an upper portion of the leg of the wearer wherein said leg sleeve covers at least a portion of the leg of the wearer;
a torso cover comprising a front facing side, a rear facing side, and an opening proximate a neck of the wearer wherein said torso cover covers at least a portion of a torso of the wearer;
a helmet wherein said helmet covers at least a portion of a head of the wearer;
a footwear wherein said footwear covers at least a portion of a foot of the wearer; and
a glove covering at least a portion of a hand of the wearer;
the arm sleeve, the leg sleeve, the torso cover, the helmet, the footwear, and the glove comprising a modular connection.

3. The bodysuit as claimed in claim 2 wherein said bodysuit is composed of a material comprising a plurality of layers;
a first, inwardly facing, layer composed of a nylon tricot material, wherein said layer is proximate the body of the wearer;
a second layer composed of spandex, adjacent to the first layer;
a third layer composed of urethane coated nylon adjacent to the second layer, wherein said third layer is able to expand and contract through a pressurization function of the bodysuit;
a fourth layer, wherein said layer is an outwardly facing layer, composed of a weaved material comprising Kevlar and graphene; and
a plurality of carbon nanotubes suspended in polyethylene glycol, wherein said carbon nanotubes are interposed within the plurality of layers.

4. The bodysuit as claimed in claim 2, wherein at least one of the following segments including the front facing side of the torso cover, the rear facing side of the torso cover, and a portion of the bodysuit proximate a waist of the wearer comprise a main control module coupled to the segment of the bodysuit; and
the bodysuit further comprising a plurality of primary central control modules wherein said primary central control modules communicate via wireless transmission to the main central control modules
the primary central control modules being coupled to the segments of the suit.

5. The bodysuit as claimed in claim 4, wherein the hardware further comprises a processor, a memory storage device, and a power supply.

6. The bodysuit as claimed in claim 5 further comprising an interactive user interface that allows wearers to interact wirelessly to a network provided by an external server via a wearable device and a mobile phone comprising an application.

7. The bodysuit as claimed in claim 4 further comprising at least one primary reserve control module.

8. The bodysuit as claimed in claim 5 wherein the hardware devices and the IO devices provide data to the main central control module to facilitate a plurality of functions.

9. The bodysuit as claimed in claim 8 wherein the plurality of functions comprise:
a first function wherein bodysuit performs a collection of biometric data of the wearer;
a second function wherein the bodysuit facilitates the uptake of nanoparticles throughout the body of the wearer;
a third function wherein the temperature of the body suit increases providing a heat therapy to the wearer;
a fourth function wherein the third layer of the multi-layered material expands thus restricting a flow of blood, and thus providing a cooling sensation, to a portion of the body of the wearer to simulate cold therapy;
a fifth function wherein the third layer of the multi-layered material expands thus compressing a portion of the body of the wearer, providing a compression therapy to the wearer;
a sixth function wherein the third layer of the multi-layered material contracts thus decompressing a portion of the body of the wearer;
a seventh function wherein the biometric sensors detect and measure electrical signals from nerves throughout the body, the processor and wireless transmitter communicate the data acquired from the biometric sensors to the primary central control module, the primary central control module transmitting the data to the main central control module, and the main central control module transmitting the data to the user interface wherein the user interface compares electrical signals throughout the body and diagnoses nerve imbalances;
an eighth function wherein the bodysuit facilitates the enhancement, repair, recovery, and rehabilitation of a muscle of a wearer;
a ninth function wherein electrical impulses are delivered through the bodysuit to increase circulation, increase mobility, assist in healing, and facilitate recovery; and
a tenth function wherein biometric reads are taken by the biometric sensors to detect and facilitate the uptake of pharmaceutical products.

10. The bodysuit as claimed in claim 9 wherein the first function comprises the steps of:
perform biometric reads of electrical impulses, oxygen saturation, blood pressure, and systematic imbalances;
monitor a plurality of biometric data over an interval of time;
record the biometric data; and
compile the data and information acquired by the biometric sensors.

11. The bodysuit as claimed in claim 9 wherein the second function comprises the steps of:
nanomino enzymatically encapsulated liquid amino acids are ingested by wearer;
release of nanomino enzymatically liquid amino acids are delayed using a prolonged release capsule;
amino acids are directed to a target area; and
bodysuit stimulates a portion of the body proximate the target area of the body of the wearer.

12. A bodysuit comprising:
at least one segment wherein said segment covers at least a portion of a body of a wearer;
the segment further comprising at least one main central control module and at least one primary central control module wherein the main central control module and the primary central control module are coupled to the segment of the bodysuit;
the at least one segment comprises at least one of the following including:
an arm sleeve comprising a first opening proximate a forearm of a wearer and a second opening proximate a shoulder of the wearer;
a leg sleeve comprising a first opening proximate a lower portion of a leg of the wearer and a second opening proximate an upper portion of the leg of the wearer wherein said leg sleeve covers at least a portion of the leg of the wearer;
a torso cover comprising a front facing side, a rear facing side, and an opening proximate a neck of the wearer wherein said torso cover covers at least a portion of a torso of the wearer;
a helmet wherein said helmet covers at least a portion of a head of the wearer;
a footwear wherein said footwear covers at least a portion of a foot of the wearer; and
a glove covering at least a portion of a hand of the wearer;
the arm sleeve, the leg sleeve, the torso cover, the helmet, the footwear, and the glove comprising a modular connection.

13. The bodysuit as claimed in claim 12 wherein said bodysuit is composed of a material comprising a plurality of layers;
a first, inwardly facing, layer composed of a nylon tricot material, wherein said layer is proximate the body of the wearer;
a second layer composed of spandex, adjacent to the first layer;
a third layer composed of urethane coated nylon adjacent to the second layer, wherein said third layer is able to expand and contract through a pressurization function of the bodysuit;
a fourth layer, wherein said layer is an outwardly facing layer, composed of a weaved material comprising Kevlar and graphene; and
a plurality of carbon nanotubes suspended in polyethylene glycol, wherein said carbon nanotubes are interposed within the plurality of layers.

14. The bodysuit as claimed in claim 13 wherein the at least one main central control module is coupled to at least one of the areas of the segments selected from a group consisting of a front facing portion of the torso cover of the bodysuit and a rear facing portion of the torso cover of the bodysuit;
the at least one main central control module comprising:
a processor;
a memory storage device;
a power supply; and
at least one input/output (IO) device wherein said device comprises a biometric sensor, an accelerometer, a gyroscope, and a wireless transmitter.

15. The bodysuit as claimed in claim 14 further comprising an interactive user interface that allows wearers to interact wirelessly to a network provided by an external server via a wearable device and a mobile phone comprising an application.

16. The bodysuit as claimed in claim 12 further comprising at least one primary reserve control module.

17. A bodysuit composed of a multi-layered material comprising:
at least one segment wherein said segment covers at least a portion of a body of a wearer;
the segment further comprising at least one main central control module and at least one primary central control module wherein the main central control module and the primary central control module are coupled to the segment of the bodysuit;
the at least one segment comprises at least one of the following including:
an arm sleeve comprising a first opening proximate a forearm of a wearer and a second opening proximate a shoulder of the wearer;
a leg sleeve comprising a first opening proximate a lower portion of a leg of the wearer and a second opening proximate an upper portion of the leg of the wearer wherein said leg sleeve covers at least a portion of the leg of the wearer;
a torso cover comprising a front facing side, a rear facing side, and an opening proximate a neck of the wearer wherein said torso cover covers at least a portion of a torso of the wearer;
a helmet wherein said helmet covers at least a portion of a head of the wearer;
a footwear wherein said footwear covers at least a portion of a foot of the wearer; and
a glove covering at least a portion of a hand of the wearer;
the arm sleeve, the leg sleeve, the torso cover, the helmet, the footwear, and the glove comprising a modular connection
the segment further comprising at least one hardware device wherein said hardware device is an input/output device such as a biometric sensor, an accelerometer, a gyroscope, and a wireless transmitter;
the hardware device coupled to the segment of the bodysuit, wherein said hardware device has the capability to wirelessly transmit data between said hardware device, the primary central control module, and the main central control module;
the material comprising a plurality of layers wherein the plurality of layers comprises:
a first, inwardly facing, layer composed of a nylon tricot material, wherein said layer is proximate the body of the wearer;
a second layer composed of spandex, adjacent to the first layer;
a third layer composed of urethane coated nylon adjacent to the second layer, wherein said third layer is able to expand and contract through a pressurization function of the bodysuit;
a fourth layer, wherein said layer is an outwardly facing layer, composed of a weaved material comprising Kevlar and graphene; and
a plurality of carbon nanotubes suspended in polyethylene glycol, wherein said carbon nanotubes are interposed within the plurality of layers.

18. The bodysuit as claimed in claim 17 further comprising an interactive user interface that allows wearers to interact wirelessly to a network provided by an external server via a wearable device and a mobile phone comprising an application.

19. The bodysuit as claimed in claim 18 further comprising at least one primary reserve control module.

20. The bodysuit as claimed in claim 19 wherein the hardware devices and the IO devices provide data to the main central control module to facilitate a plurality of functions including a function wherein sound waves are used to communicate information.
